(19) United States
(12) Patent Application Publication
(10) Pub. No.: US 2025/0020638 A1
(43) Pub. Date: Jan. 16, 2025

(54) Pregnancy Indicator

(71) Applicant: Proteus Zolia, Lynn, MA (US)

(72) Inventor: Proteus Zolia, Lynn, MA (US)

(21) Appl. No.: 18/769,052

(22) Filed: Jul. 10, 2024

Related U.S. Application Data
(60) Provisional application No. 63/513,288, filed on Jul. 12, 2023.

(51) Int. Cl. G01N 33/53

(52) U.S. Cl. CPC G01N 33/5302; G01N 2333/59

(57) ABSTRACT

A pregnancy indicator having the form and shape of a human female figurine. Upon determining a positive result, the pregnancy indicator provides an auditory output and a visual output. Upon a positive result, the visual output includes inflation of the breasts and abdomen of the figurine, simulating the biological changes experienced by a female during pregnancy. Additionally, the auditory output includes an audio statement saying, “I am pregnant” upon a positive result. Contrarily, upon determining a negative result, the pregnancy indicator provides an auditory output stating “I am not pregnant.” The pregnancy indicator provides a visual and auditory indication to the user, thus preventing confusion and ambiguity in the test results.

### FIELD OF THE INVENTION

The present invention relates generally to an indicator for detecting pregnancy. More specifically, the present invention is a pregnancy test that mimics the bodily transformation of pregnancy to indicate a positive test and thus eliminates ambiguity of the test results.

### BACKGROUND OF THE INVENTION

Pregnancy tests are commonly used to determine pregnancy status, primarily relying on test strips that detect human chorionic gonadotropin (hCG) hormone in urine. However, these tests often suffer from limitations, including ambiguous results and difficulties in interpretation. The prior art is capable of detecting the presence of the human chorionic gonadotropin (hCG) hormone, which is produced during pregnancy, to determine if a woman is pregnant or not. Traditional pregnancy tests typically use test strips that react to hCG in urine. These strips contain antibodies that bind to the hormone and produce visible lines or symbols as indicators of a positive result. However, these test strips often present challenges in interpretation. Ambiguous results, faint lines, or the absence of clear indicators can lead to confusion and uncertainty for users. In some cases, the visual cues may be difficult to discern, especially for individuals with visual impairments or those unfamiliar with the test format. Furthermore, conventional pregnancy tests primarily rely on visual indicators, neglecting other sensory cues that could enhance the comprehensibility of the result. This limitation can make the testing process less user-friendly and inclusive.

In all, traditional pregnancy tests employ test strips to detect hCG hormone in urine, but they often suffer from ambiguity and limited comprehensibility. The present invention addresses these pitfalls by incorporating a female figurine design with a slot for the test strip, an actuator for simulating pregnancy changes, and auditory outputs for clearer result indication. These innovations aim to provide a more user-friendly, inclusive, and comprehensive pregnancy testing experience.

One objective of the present invention is to address these limitations and provide a pregnancy test with a more comprehensive and understandable indication. To achieve this, the invention incorporates the test into a female figurine. The figurine features a dedicated slot designed to securely hold a lateral flow test strip, ensuring accurate and reliable testing. By integrating the test strip within the figurine, the invention merges functionality with aesthetics, enhancing user engagement and result comprehension. Additionally, another objective of the present invention is to indicate a positive test by simulating pregnancy within the figurine itself. When a positive test result is detected, an actuator within the figurine inflates the breast and stomach areas, mimicking the physical changes associated with pregnancy. This visual and tactile simulation provides users with a tangible representation of the pregnancy indication, facilitating a deeper connection and understanding of the result. In addition to visual and tactile cues, the invention incorporates auditory outputs as supplementary indicators. A positive test triggers an auditory output signaling the presence of pregnancy, while a negative test prompts a different auditory output indicating its absence. These auditory cues contribute to a multi-sensory experience, enhancing overall comprehension and result clarity. The figurine-based design offers distinct advantages over traditional tests. It provides a more intuitive and holistic testing experience, leveraging visual, tactile, and auditory cues for effective result communication. This approach reduces ambiguity, promotes user understanding, and instills confidence in the test outcome. Moreover, the figurine design enhances accessibility and inclusivity. Individuals with visual impairments or difficulty interpreting traditional test strip indicators benefit from the tangible and sensory nature of the invention. The combination of visual, tactile, and auditory feedback enables accurate determination of pregnancy status across a range of user abilities. In summary, the present invention revolutionizes pregnancy testing by introducing a female figurine-based design. Through the integration of a test strip slot, an actuator for simulating pregnancy changes, and auditory outputs, the invention provides a comprehensive and user-friendly testing experience. Its innovative approach enhances clarity, reduces ambiguity, and promotes inclusivity in the process of determining pregnancy status.

### SUMMARY OF THE INVENTION

The invention is a pregnancy test designed as a female figurine. It provides a clear and easy-to-understand indication of pregnancy. The figurine has a slot for inserting a special test strip, ensuring accurate results. When the test shows a positive result, the figurine's breast and stomach areas inflate, mimicking pregnancy. It also plays a specific sound to indicate a positive or negative result. The figurine design is especially helpful for people with visual difficulties. Overall, the female figurine pregnancy test offers a comprehensive, enjoyable, and reliable way to know if you're pregnant or not.

### BRIEF DESCRIPTION OF THE DRAWINGS

FIG. 1 is a front view diagram of the present invention.

FIG. 2 is a front view diagram of the present invention, further including internal components.

FIG. 3 is a tabular diagram of the figurine of the present invention.

FIG. 4 is a diagram of the present invention detailing the test strip insertion port, the processing unit, and user indicator system.

FIG. 5 is a diagram of the user indicator system.

FIG. 6 is a detailed diagram of the present invention further including a process and circuit diagram.

FIG. 7 is a process diagram of the computer implemented method of the present invention.

FIG. 8 is a process diagram of the computer implemented method, specifically detailing the positive indication of hCG.

FIG. 9 is a process diagram of the computer implemented method, specifically detailing the negative indication of hCG.

FIG. 10 is a dual-profile view of the present invention comprising a pump showing the deflated configuration (left) and the inflated configuration (right).

FIG. 11 is a dual-profile view of the present invention comprising an actuator showing the deflated configuration (left) and the inflated configuration (right).

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a pregnancy indicator 1, embodiments of the present disclosure are not limited to use only in this context.

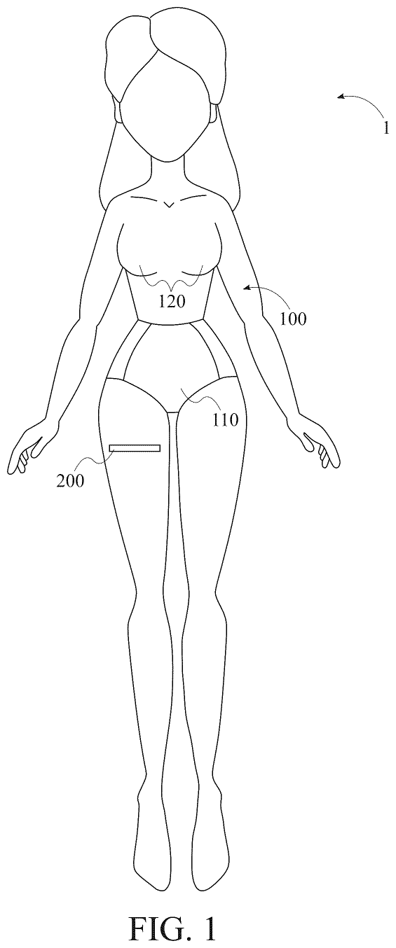

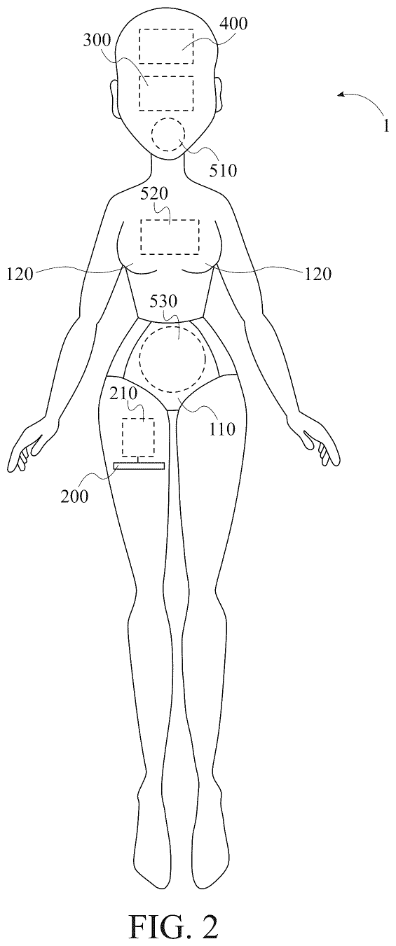

As shown in FIG. 1, the present invention is a pregnancy indicator 1 comprising a figurine 100 and a test strip insertion port 200. Within the context of the present invention, the figurine 100 is a human shaped figurine and is to be understood as comprising body parts in a manner resembling a human female. Therefore, when body parts, such as head, chest, back, abdomen, legs, arms, torso, etc. are mentioned, it is assumed and understood that the body part is used in its plain and routine meaning, unless otherwise stated. In the preferred embodiment of the present invention, the test strip insertion port 200 is integrated (embedded) into the figurine 100 wherein a hCG test strip 2 may be inserted into the test strip insertion port 200 and thereby being inserted into the figurine 100. Furthermore, the figurine 100, as shown in FIG. 1 and FIG. 2, comprises an inflatable chest 120 and an inflatable abdomen 110. Additionally, in the preferred embodiment of the present invention, the figurine 100, specifically the inflatable chest 120 and inflatable abdomen 110, are composed of an elastic material, such as rubber. In the context of the present invention, the inflatable chest 120 is composed of two features resembling female breasts. Furthermore, within the context of the present invention, the term “inflatable” is expanded to include “expandable” and “malleable.”

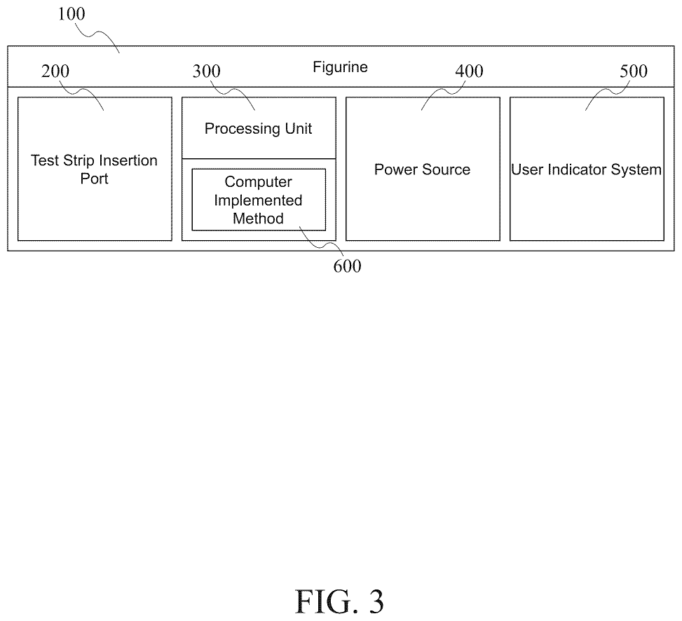

Furthermore, as shown in FIG. 2, the figurine 100 further houses a processing unit 300 and a power source 400. In the preferred embodiment, the power source 400 is a battery. Moreover, in the context of the present invention, the power source 400 provides power to the components of the present invention. Further, the processing unit 300 executes instructions of a computer program, such as arithmetic, logic, controlling, and input/output (I/O) operations. Additionally, as shown in FIG. 2 and FIG. 3, the figurine 100 further houses a user indictor system 500 (shown in FIG. 3) composed of a speaker 510, a chest motion system 520, and an abdominal motion system 530. In the present invention, the chest motion system 520 is disposed within the figurine 100, beneath the inflatable chest 120. Likewise, the abdominal motion system 530 is disposed within the figurine 100, beneath the inflatable abdomen 110. Additionally, as shown in FIG. 2, the test strip insertion port 200 comprises a lateral flow reader 210, wherein the lateral flow reader 210 is contained within the figurine 100. As shown in FIG. 3, the processing unit 300 executes a computer implemented method 600 wherein said computer implemented method 600 performs commands within the present invention in order to indicate to the user, the presence of human chorionic gonadotropin (hCG), and as a result, pregnancy status. Furthermore, it may be said that the abdominal motion system 530 and the chest motion system 520, each distort the inflatable abdomen 110 and inflatable chest 120, respectively, to resemble the shape of a pregnant human female upon indication of human chorionic gonadotropin (hCG).

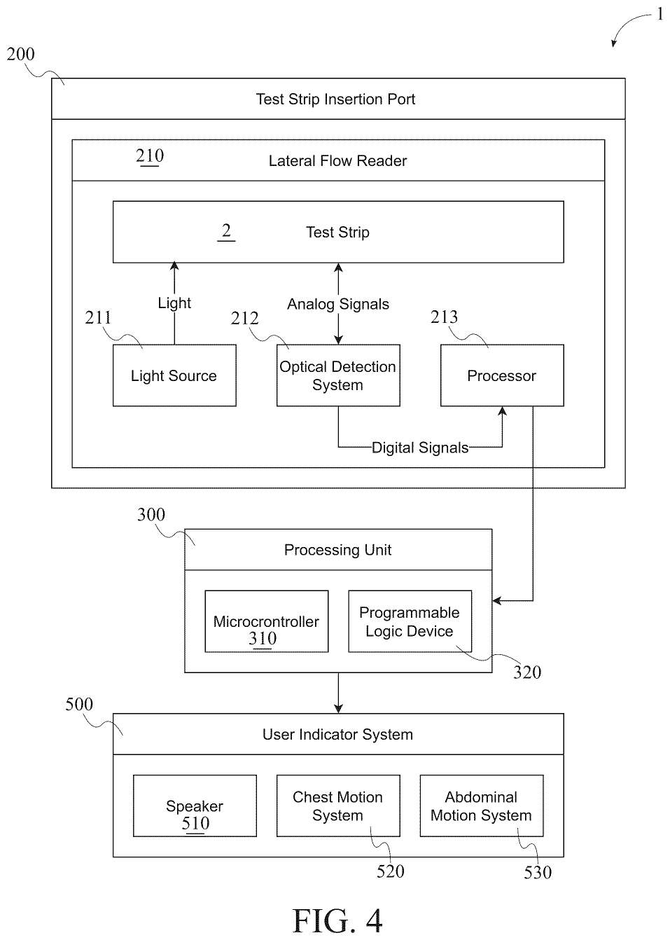

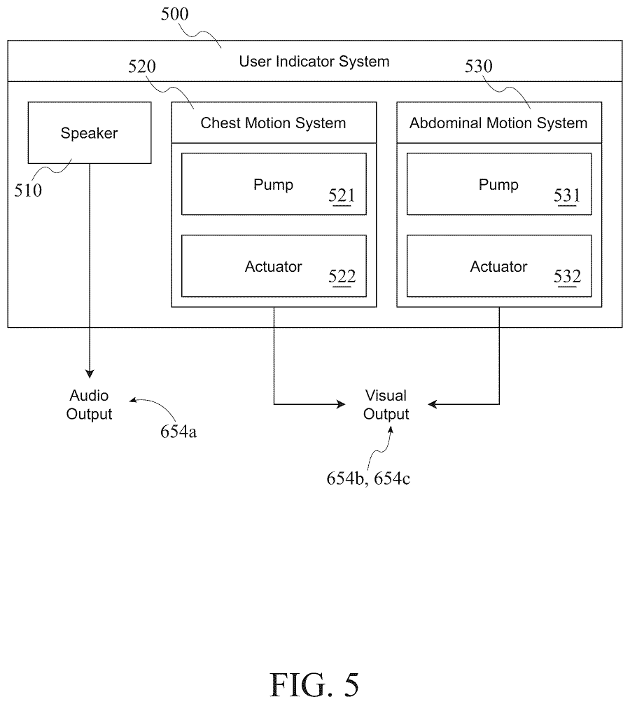

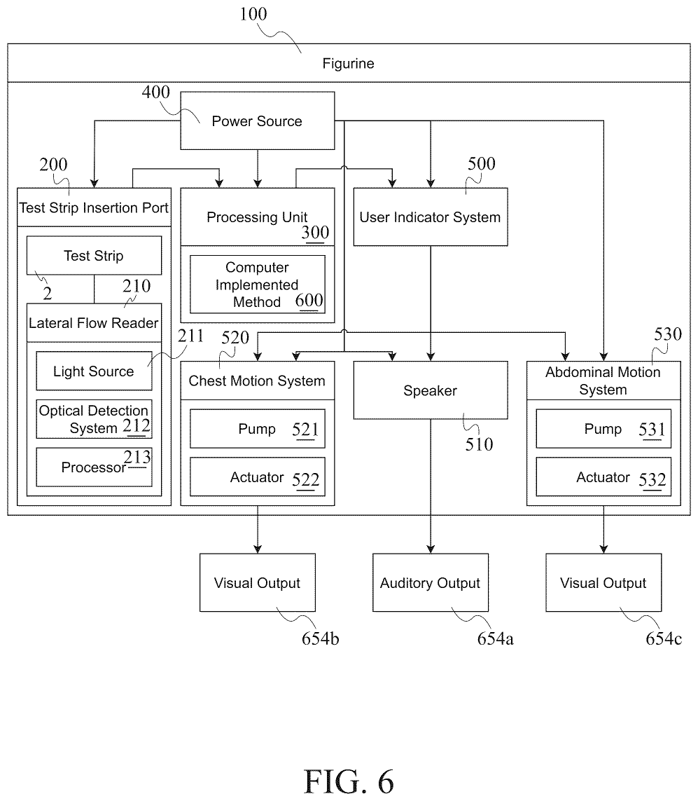

In the preferred embodiment of the present invention, as shown in FIG. 4, a test strip 2 is inserted into the pregnancy indicator 1, via the test strip insertion port 200, whereby the test strip 2 is received into the lateral flow reader 210. The lateral flow reader 210 comprises a light source 211, an optical detection system 212, and a processor 213. In the present invention, the light source 211 illuminates the test strip 2, wherein the optical detection system 212 receives sensory input in the form of analog signals from the light emitted from the test strip 2. The light source 211 of the present invention may comprise an LED or a laser diode that emits light. The optical detection system 212, which may comprise photodetectors that measure the intensity of emitted light captures the optical signals emitted by the test strip 2. Further, the optical detection system 212 then converts the analog signals into digital signals and sends the digital signals to the processor 213. The processor 213 communicates data from the lateral flow reader 210 to the processing unit 300, wherein the processing unit 300 comprises a microcontroller 310 and a programmable logic device 320. The microcontroller 310 and the programmable logic device 320 receive the data from the processor 300, processing said data to determine the presence of hCG on the test strip 2. Once the data is processed, a signal is sent to the user indicator system 500, comprising the speaker 510, the chest motion system 520, and the abdominal motion system 530, wherein an output is generated to inform the user of the pregnancy status. As shown in FIG. 5 and FIG. 6, the speaker 510 provides an audio/auditory output 654a, wherein the chest motion system 520 and the abdominal motion system 530, each provide a visual output 654b,654c. In the preferred embodiments of the present invention, each of the chest motion system 520 and the abdominal motion system 530 may comprise a pump 521,531, an actuator 522,532, or a combination thereof. Additionally, as shown in FIG. 6, the power source 400 provides power to the test strip insertion port 200, the processing unit 300, and the user indicator system 500.

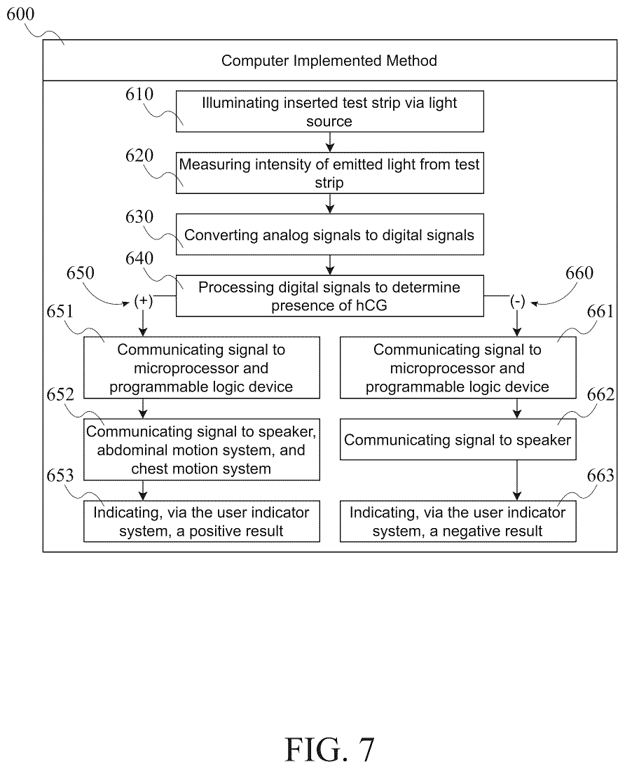

As shown in FIG. 7, the computer implemented method 600 of the present invention comprises the steps:
610. illuminating inserted test strip via the light source of the lateral flow reader;
620. measuring the intensity of light emitted from the test strip;
630. converting analog signals to digital signals; and
640. processing signals to determine the presence of hCG;
650. IF there is a positive presence of hCG:
   651. communicating signal to microprocessor and programmable logic device;
   652. communicating signal to speaker, abdominal motion system; and chest motion system; and
   653. indicating, via the user indicator system, a positive result;
660. ELSE if there is a negative presence of hCG:
   661. communicating signal to microprocessor and programmable logic device;
   662. communicating signal to speaker; and
   663. indicating, the user indicator system, a negative result.

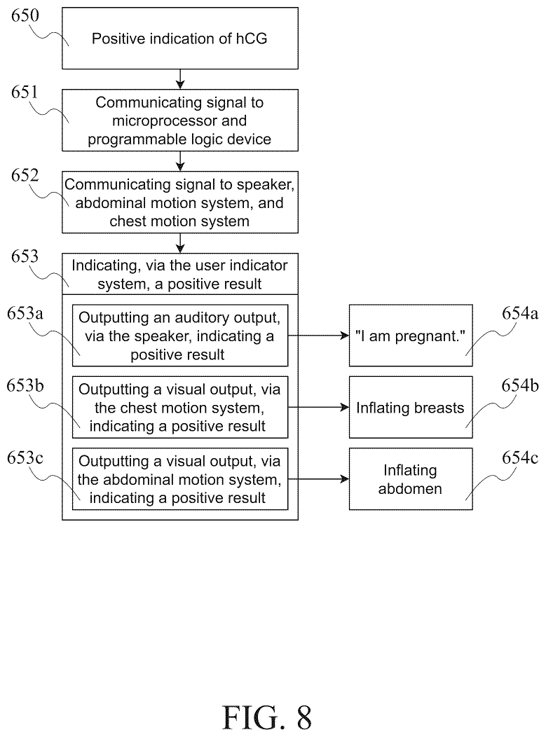

Furthermore, as shown in FIG. 8, during a positive presence of hCG, upon indicating, via the indicator system, a positive result, the computer implemented method further comprises the steps of:
653a. outputting an auditory output, via the speaker, indicating a positive result;
   654a. the auditory output is an audible “I am pregnant;”
653b. outputting via a visual output, via the chest motion system, indicating a positive result;
   654b. the visual output is the inflation of the inflatable chest; and
653b. outputting via a visual output, via the abdominal motion system, indicating a positive result;
   654b. the visual output is the inflation of the inflatable abdomen.

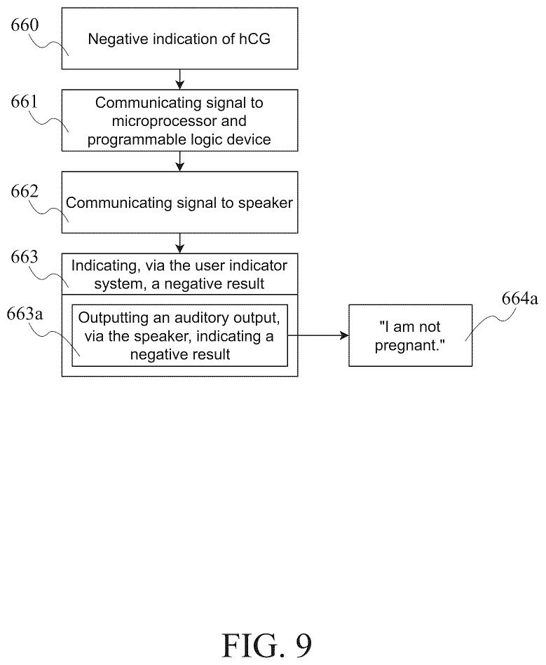

As shown in FIG. 9, upon determination of a negative indication of hCG, the computer implemented method 600 further comprises the steps of:
663a. outputting an auditory output, via the speaker, indicating a negative result;
   654a. the auditory output is an audible “I am not pregnant.”

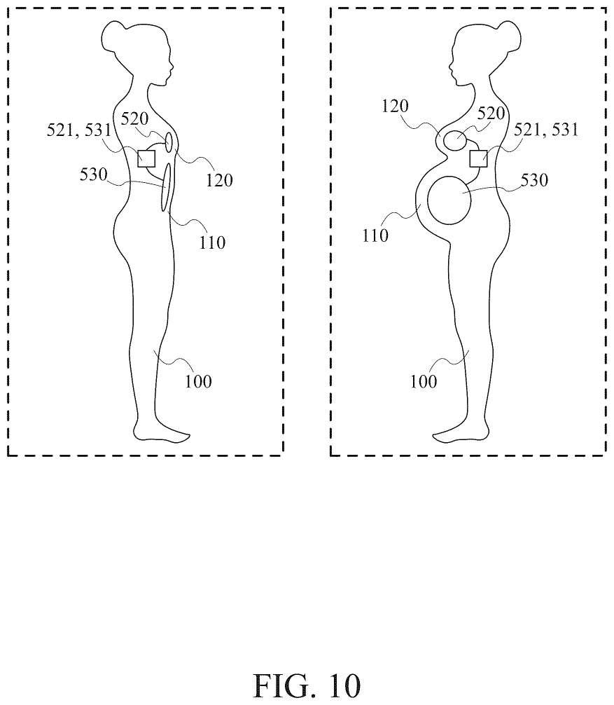

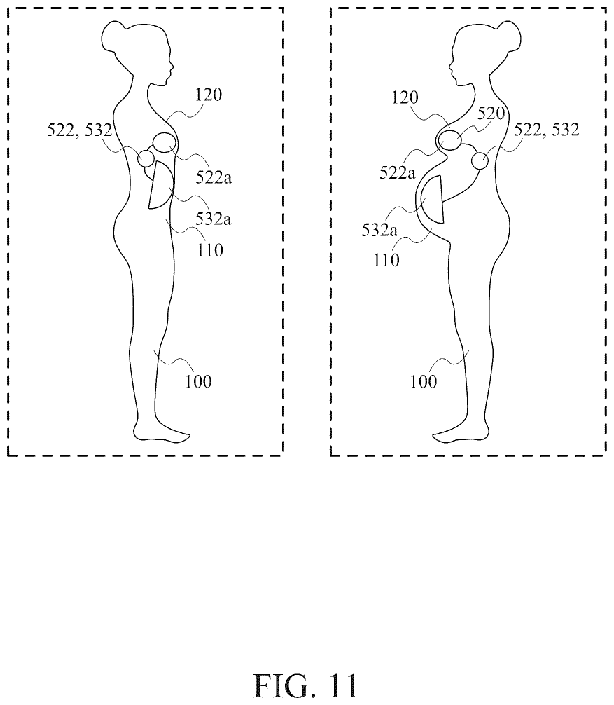

As previously disclosed and as further illustrated in FIG. 10 and FIG. 11, in the preferred embodiment of the present invention, the chest motion system 520 and the abdominal motion system 530 may each comprise either the pump 521,531 or the actuator 522,532. In embodiments wherein the chest motion system 520 or the abdominal motion system 530 comprises a pump 521,531, as shown in FIG. 10, each comprises an inflatable cavity disposed behind the inflatable chest 120 and the inflatable abdomen 110, respectively. In such embodiments, the pump 521,531 forces air into the cavities, thus inflating the figurine 100 to resemble a pregnant female figure. Contrarily, in embodiments wherein an actuator 522,532 is utilized, the figurine 100 comprises a chest shell 522a and an abdominal shell 532a, as shown in FIG. 11. In such embodiments, as shown in FIG. 11, the chest shell 522a and the abdominal shell 532a, located within the inflatable chest 120 and inflatable abdomen 110, respectively, are rigid members that, when indicating a positive result, are forced, by the actuator 522,532, against the inflatable chest 120 and inflatable abdomen 110, respectively, thus forming the appearance of a pregnant human female.

In some alternative embodiments of the present invention, the figurine 100 may be configured to comprise clothing garments. In such embodiments, the clothing garments, specifically the portion of clothing garments adjacent to the inflatable chest 120 and inflatable abdomen 110, are preferably composed of elastic material. Furthermore, in some embodiments wherein the figurine 100 is clothed, the inflatable chest 120 and inflatable abdomen 110 may be partially composed of the clothing garment. In such embodiments, this may be a cost effective approach to concealing the internal mechanisms of the present invention.

Furthermore, herein, the test strip 2, as understood to those in the art is a standard hCG test strip.

Although the invention has been explained in relation to its preferred embodiment, it is to be understood that many other possible modifications and variations can be made without departing from the spirit and scope of the invention.

1. A pregnancy indicator comprising a figurine having an inflatable abdomen and an abdominal motion system;
the inflatable abdomen being composed of an elastic material;
the abdominal motion system being disposed within the figurine adjacent to the inflatable abdomen; and
the abdominal motion system distorting the inflatable abdomen to resemble the shape of a pregnant human female upon indication of a human chorionic gonadotropin presence.

2. The pregnancy indicator as claimed in claim 1, further comprising a test strip insertion port;
the test strip insertion port comprising a lateral flow reader; and
the lateral flow reader receiving a human chorionic gonadotropin (hCG) test strip.

3. The pregnancy indicator as claimed in claim 2, wherein the lateral flow reader comprises:
a light source;
an optical detection system; and
a processor
wherein:
the light source illuminates the hCG test strip;
the optical detection system measures the light emitted from the test strip and;
the processor receives digital signals from the optical detection system thus indicating the presence of hCG.

4. The pregnancy indicator as claimed in claim 3, further comprising a processing unit;
the processing unit comprising a microcontroller and a programmable logic device.

5. The pregnancy indicator as claimed in claim 4 wherein the processing unit executes a computer implemented method comprising the steps of:
illuminating the hCG test strip via the light source of the lateral flow reader;
measuring the intensity of light emitted from the test strip, via the optical detection system; and
processing signals to determine the presence of hCG, via the processor.

6. The pregnancy indicator as claimed in claim 5, wherein, in the presence of hCG, the computer implemented method further comprises the steps of:
communicating a signal to the microprocessor and programmable logic device;
communicating a signal to the abdominal motion system; and
indicating, via the abdominal motion system, a positive result.

7. The pregnancy indicator as claimed in claim 6, wherein, upon indicating, via the abdominal motion system, a positive result, the abdominal motion system outputs a visual output indicating a positive result.

8. The pregnancy indicator as claimed in claim 7, wherein the visual output indicated by the abdominal motion system is an inflation of the inflatable abdomen.

9. The pregnancy indicator, as claimed in claim 6, further comprising an inflatable chest and a chest motion system;
the inflatable chest being composed of an elastic material;
the chest motion system being disposed within the figurine adjacent to the inflatable chest; and
the chest motion system distorting the inflatable chest to resemble the shape of a pregnant human female breasts upon indication of a hCG presence.

10. The pregnancy indicator as claimed in claim 9, wherein, upon indicating, via the chest motion system, a positive result, the chest motion system outputs a visual output indicating a positive result.

11. The pregnancy indicator as claimed in claim 10, wherein the visual output indicated by the chest motion system is an inflation of the inflatable chest.

12. The pregnancy indicator as claimed in claim 6 further comprising a speaker.

13. The pregnancy indicator as claimed in claim 12, wherein, upon indicating, via the speaker, a positive result, the speaker outputs a auditory output indicating a positive result.

14. The pregnancy indicator as claimed in claim 13, wherein the auditory output indicated by the speaker is a verbal “I am pregnant.”

15. The pregnancy indicator as claimed in claim 5, further comprising a speaker, wherein, to indicate a negative result, the computer implemented method further comprises the steps of:
communicating a signal to the microprocessor and programmable logic device;
communicating a signal to the speaker; and
indicating, via an auditory output by the speaker, a negative result;
wherein:
the auditory output is a verbal “I am not pregnant.”

16. A pregnancy indicator comprising a figurine having an inflatable abdomen, an abdominal motion system, and a speaker;
the inflatable abdomen being composed of an elastic material;
the abdominal motion system being disposed within the figurine adjacent to the inflatable abdomen; and
the abdominal motion system distorting the inflatable abdomen to resemble the shape of a pregnant human female upon indication of a human chorionic gonadotropin presence.

17. The pregnancy indicator as claimed in claim 16, further comprising a test strip insertion port and a processing unit;
the test strip insertion port comprising a lateral flow reader;
the lateral flow reader comprising
a light source;
an optical detection system; and
a processor
wherein:
the light source illuminates the hCG test strip;
the optical detection system measures the light emitted from the test strip;
the processor receives digital signals from the optical detection system thus indicating the presence of hCG; and
the lateral flow reader receiving a human chorionic gonadotropin (hCG) test strip.

18. The pregnancy indicator as claimed in claim 17 wherein the processing unit executes a computer implemented method comprising the steps of:
illuminating the hCG test strip via the light source of the lateral flow reader;
measuring the intensity of light emitted from the test strip, via the optical detection system; and
processing signals to determine the presence of hCG, via the processor.

19. A pregnancy indicator comprising:
a figurine;
a test strip insertion port;
a processing unit; and
a user indication system;
the figurine comprising an inflatable abdomen;
the test strip insertion port being embedded in the figurine;
the test strip insertion port comprising a lateral flow reader wherein said lateral flow reader receives a hCG test strip;
the processing unit executing a computer implemented method;
the user indication system comprising an abdominal motion system;
the abdominal motion system being disposed within the figurine adjacent to the inflatable abdomen; and
the abdominal motion system distorting the inflatable abdomen to resemble the shape of a pregnant human female upon indication of a human chorionic gonadotropin presence.

20. The pregnancy indicator as claimed in claim 5, wherein, in the presence of hCG, the computer implemented method further comprises the steps of:
communicating a signal to the microprocessor and programmable logic device;
communicating a signal to the abdominal motion system;
indicating, via the abdominal motion system, a positive result;
upon indicating the positive result, the abdominal motion system outputs a visual output indicating a positive result;
the visual output indicated by the abdominal motion system is an inflation of the inflatable abdomen.
